(19) United States
(12) Patent Application Publication
(10) Pub. No.: US 2014/0150807 A1
(43) Pub. Date: Jun. 5, 2014

(54) RESTRAINT AND CONTAINMENT SYSTEMS

(71) Applicant: Aegis Industries, Inc., Rockville, MD (US)

(72) Inventors: Kenneth J. Stethem, Bellevue, ID (US); Stephen Simantiras, Morristown, NJ (US)

(73) Assignee: Aegis Industries, Inc., Rockville, MD (US)

(21) Appl. No.: 13/890,428

(22) Filed: May 9, 2013

Related U.S. Application Data
(60) Provisional application No. 61/644,895, filed on May 9, 2012.

(51) Int. Cl. A61F 5/37

(52) U.S. Cl. CPC A61F 5/3723
USPC 128/870

(57) ABSTRACT

A restraint system includes a first handle and a second handle. A sling element is connected at a first edge to the first handle and at a second edge to the second handle and includes a webbed material.

### CROSS-REFERENCE TO RELATED APPLICATIONS

This application claims priority to and the benefit of U.S. Provisional Patent Application Ser. No. 61/644,895, filed May 9, 2012, entitled “Restraint and Containment Systems,” the disclosure of which is hereby incorporated by reference herein in its entirety.

### INTRODUCTION

Restraint devices such as straightjackets, handcuffs, ropes, shackles, wrist restraints, etc., may be used to restrain or otherwise restrict movement of an individual. Depending on the device modality, the device may be used to secure a portion of the individual (e.g., handcuffs secure hands and wrists) or the individual generally (e.g., straightjackets secure the entire upper body and appendages). Use of such devices may be desirable in military and law enforcement applications to secure combatants and criminal suspects, and in the medical field to secure mentally or emotionally unstable individuals. Regardless, there is risk inherent the use of these devices to both the target individual and the device user. For example, to attach handcuffs or a straightjacket, the device user must first overpower the target, which may be exceedingly difficult if the individual is violent or otherwise physically uncooperative.

### SUMMARY

In one aspect, the technology relates to a restraint system having: a first handle and a second handle; and a sling element connected at a first edge to the first handle and at a second edge to the second handle, the sling element including a webbed material. In an embodiment, the sling element further includes a first non-webbed section connected to the first handle and the webbed material. In another embodiment, the sling element further includes a second non-webbed section connected to the second handle and the webbed material. In yet another embodiment, the sling element includes an outer face and an inner face. In still another embodiment, the sling element inner face includes a pouch securement area including a first securement area and a second securement area.

In an embodiment of the above aspect, the restraint system includes a pouch fixed to the pouch securement area. In another embodiment, the pouch includes: a first securement portion; a second securement portion, wherein each of the first securement portion and the second securement portion has an outer face and an inner face; and a fold located between the first securement portion and the second securement portion. In another embodiment, the outer face of the first securement portion is connected to the first securement area and the outer face of the second securement portion is connected to the second securement area. In yet another embodiment, the inner face of the first securement portion and the inner face of the second securement portion are adapted to be connected to each other when the restraint system is in a stored position and disconnected from each other when the restraint system is in a deployed position. In still another embodiment, the pouch is adapted to receive a distraction element, wherein the distraction element is adapted to be discharged from the pouch when the restraint system is in the deployed position.

In an embodiment of the above aspect, the distraction element includes at least one of glitter, frag, and powder. In another embodiment, when in the deployed position, the sling element is taut between the first handle and the second handle. In another embodiment, the restraint system includes a third handle connected to the sling element outer face. In yet another embodiment, the sling element outer face includes at least one accessory fixture. In still another embodiment, the accessory fixture includes at least one of a snap connection, a pocket, an elastic element, and a loop. In another embodiment, the restraint system further includes a connecting element proximate the first handle, wherein the connecting element is adapted to connect the restraint system to a second restraint system.

In another aspect, the technology relates to a method of deploying a restraint system having a first handle, a second handle, and a sling element strung between the first handle and the second handle, the method including: pulling the first handle in a direction substantially away from the second handle, such that the sling element is made taut between the first handle and the second handle. In another embodiment, the first handle is grasped by a first user and the second handle is grasped by a second user. In another embodiment, the restraint system automatically discharges a distraction element as the sling element is made taut. In another embodiment, the method includes wrapping the restraint system around at least one target.

### BRIEF DESCRIPTION OF THE DRAWINGS

There are shown in the drawings, embodiments which are presently preferred, it being understood, however, that the technology is not limited to the precise arrangements and instrumentalities shown.

FIG. 1 depicts an outer face of an embodiment of a restraint system.

FIG. 2 depicts an inner face of the restraint system of FIG. 1.

FIG. 3 depicts an inner face of another embodiment of a restraint system.

FIGS. 4A-4C depict perspective views of an embodiment of a distraction element pouch.

FIG. 5 depicts a partial enlarged view of an inner face of an embodiment of a restraint system.

FIGS. 6A-6F depict a sequence for using a restraint system.

### DETAILED DESCRIPTION

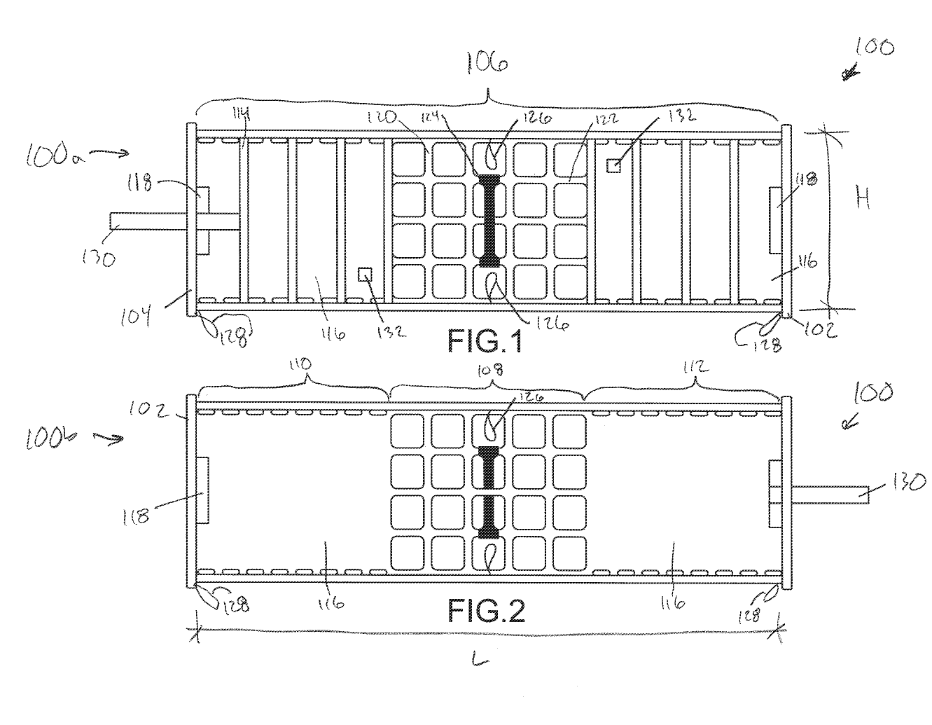

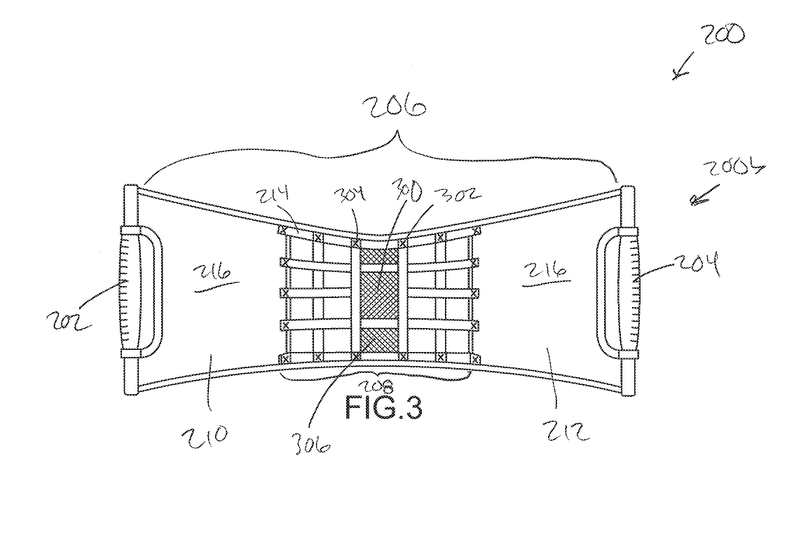

FIGS. 1 and 2 depict an outer face 100a and an inner face 100b, respectively, of a restraint system 100. The restraint system 100 includes a plurality of handles 102, 104 with a sling element 106 located therebetween. The handles 102, 104 may be removable from the system 100 so as to be employed as weapons to provide a defensive/standoff capability for the user(s). A central portion 108 of the sling element 106 may be an open mesh, webbing, or other material. Outer portions 110, 112 of the sling element 106 may be a solid fabric or other material. In certain embodiments, webbing 114 may be located along the entire length L and height H of the system 100. A solid material 116, e.g., woven polypropylene or other tear-resistant material, may span this webbing 114 on the outer portions 110, 112 of the sling 106. In certain embodiments, the webbing 114 is exposed on the outer face 110a of the sling 106, thus providing a smooth inner face 100b, which helps prevent a target from gripping the webbing 114 during deployment of the restraint system 100. In other embodiments, the solid material may be woven through the webbing. It may be advantageous to stitch the solid material 116 to the webbing 114.

In an alternative embodiment, the entire sling element 106 may be either of a webbing or solid material. Embodiments of the system 100 that include both webbing 114 and solid material 116 (that is, that have both open areas and solid areas), as depicted, however, may have functional advantages as described below. The sling element 106 is connected to the handles 102, 104 such that openings 118 are present along a portion of the handle 102, 104, in this case, the central portion. This enables a user to easily grip the handles 102, 104 for deployment of the system 100. In other embodiments, multiple openings may be present, giving the user multiple locations at which to grab the handles. The sling element 106 (or more specifically, the webbing 114 and/or solid material 116) may be looped around the handles 102, 104 and stitched, adhered, or otherwise secured to form a robust connection. Alternatively, the edges of the sling element may be secured to the handle with eyelet/loop connections, or portions of the edges may be passed through openings defined by the handles and knotted or otherwise secured to prevent removal thereof.

The webbing 114 includes vertical 120 and horizontal 122 web elements. The vertical 120 and horizontal 122 elements may be secured to each other at intersections or be allowed to hang loose. In the central portion 108, the webbing 114 may be spaced so as to allow the passage of the hands and/or arms of a target individual. Using an open webbing 114 (that is, without any solid material 116) in the central portion 108 of the sling element 106 performs a number of functions. For example, the use of webbing 114 allows arms, hands, or other appendages to pass through the sling 106. This helps leave these appendages accessible so handcuffs, wrist restraints, or other securement devices may be used to further restrain the target individual once that individual is subdued by the system 100. Additionally, weapons wielded by the target may pass through the webbing 114, allowing users to more safely disarm an armed target. Leaving the limbs exposed also provides the restraint system 100 users access to the limbs so as to ease the administration of intravenous sedatives, medicines, etc. Additionally, as the system 100 is deployed against a target individual, the webbing 114 may give the target individual the impression that there is a reduced threat in the center portion 108 of the sling element 106 or that the center portion 108 is compromised. This instinctually compels the target individual to approach the webbing 114 in an effort to escape containment by the system 100. This tactic will fail, however, due to the strength of the webbing 114. Additionally, the solid material 116 on the outer portions 110, 112 helps shield the users during use of the device 100 from an attack by the target.

A third handle 124, in the form of a rigid shaft or flexible strap is secured to the outer face 100a of the sling element 106. The handle 124 may be used to help maneuver or transport the target individual once contained in the restraint system 100. Additional handles may be located elsewhere along the sling element 106. One or more loops 126 may be attached to the sling element 106 to provide additional control points for the users of the system 100. Loops attached proximate the handles 102, 104 may be used to join multiple restraint systems together at the handles, or a single connecting element or strap 130, as depicted, may serve this purpose. This would enable multiple restraint systems to be used as a portable, flexible barrier or wall for crowd control or other purposes. Of course, a single restraint system 100 may also be used for this purpose.

The restraint systems 100 described herein are modular in nature and may be fastened in multiple sections ranging in widths from about three feet to about 50 feet or more, depending on the length L of the individual restraint systems 100. The restraint system 100 may be deployed individually, in tandem, or in multiples against an individual(s) or crowds and from the front, side or rear of the same. Loops 128 located proximate bottom portions of each handle 102, 104 may serve as foot engagement devices, such that the user may place his foot into the loops 128 to hold steady the lower portion of the handle 102, 104 while using his hands to position the upper portion of the handle 102, 104. This may be particularly useful when the restraint system 100 is being used as a portable fence or barrier for, e.g., crowd control applications. Multiple restraint systems 100 may also be used to wrap around and contain a plurality of target individuals. The connecting element or strap 130 may also be used to secure the restraint system 100 in the closed position. This strap 130 may also be used to a handle of a first system to attach a handle of an adjacent, second system. In that regard, the modular nature of the systems 100 depicted herein allow a plurality of systems 100 to be joined together as required or desired for a particular application. Accessory-holding devices 132 may be secured to accessory locations on the outer face 100a of the restraint system 100. These devices 132 may be snap connections, pockets, elastics elements, or loops. The accessory holding devices 132 may be configured to hold hand cuffs, mace canisters, or other devices that may be useful to the users. In certain embodiments, the system 100 may be stored in a stuff bag. The stuff bag and/or cords located thereon may be attached to the system 100 during deployment and used in a manner similar to the loops 126, 128, for example.

FIG. 3 depicts an alternative embodiment of a restraint system 200, specifically, an inner face 200b thereof. This embodiment of the system 200 includes many of the components of the system 100 depicted in FIGS. 1 and 2. These include handles 202, 204, a webbing 214 in the central portion 208, and solid material 216 at the outer portions 210, 212. Additionally, the depicted system 200 includes a centrally-located accessory securement area 300, reinforced, in this case, with a semi-rigid nylon mesh 306. The accessory securement area 300 includes a first securement element 302 and a second securement element 304 and is adapted to hold or otherwise secure a pouch 400 that holds a distraction element.

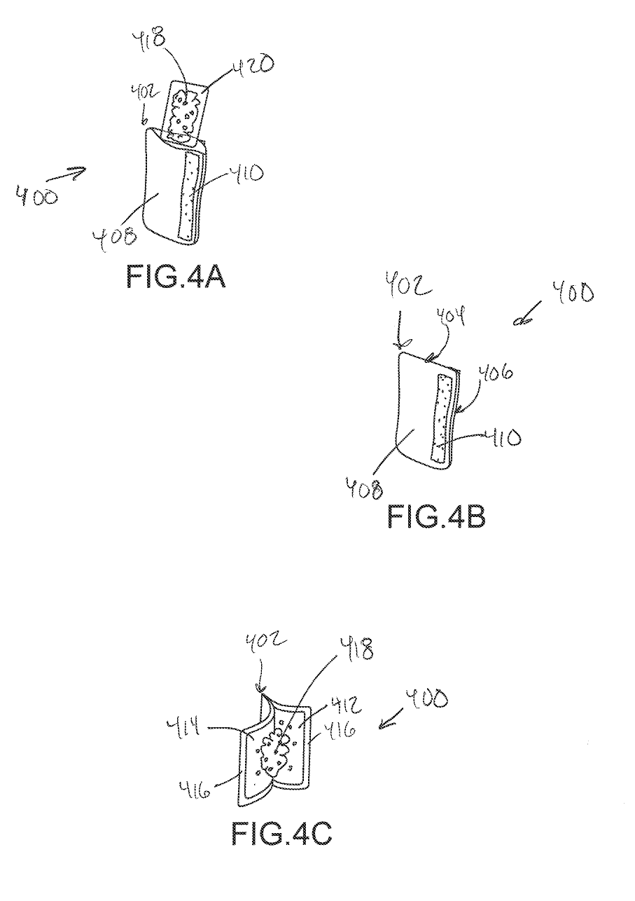

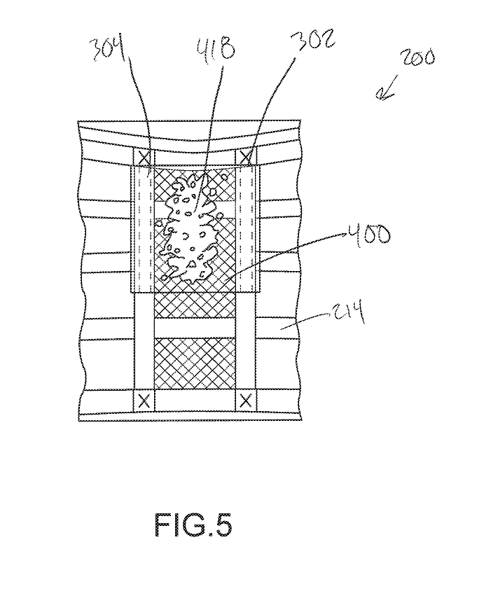

The pouch 400 is depicted in FIGS. 4A-4C and 5. The pouch 400 may be a folded fabric, plastic, or other material. The pouch 400 includes a fold 402, thus forming two outer faces and two inner faces. One outer face 404 includes a first attachment portion 406 and the other outer face 408 includes a second attachment portion 410. The inner faces 412, 414 are oriented facing each other when the pouch 400 is in a closed position (FIGS. 4A, 4B) and may be joined at their edges 416 with a hook and loop fastener (e.g., VELCRO™). The first attachment portion 406 is secured to the first securement area 302 of the system 200 and the second attachment portion 410 is secured to the second attachment area 304 of the sling element. The outer faces 404, 408 may also be further secured to the accessory securement area 300. The connections may be made with hook and loop fasteners, buttons, snaps, stitching, etc. In certain embodiments, it may be desirable for the securement areas 302, 304 to be more robustly secured to the attachment portions 406, 410 of the pouch 400, while the edges 416 are less robustly secured to each other. For example, the pouch 400 may be secured to the securement areas 302, 304 by stitching, while the edges 416 may be secured with a small area of VELCRO. This allows the pouch 400 to be deployed during use, without being inadvertently disconnected from the system 200.

The pouch 400 may contain one or more distraction, disorientation, deterrent, or disabling elements 418. These elements 418 have a detrimental effect on one or more senses of a target. They may be visual deterrents (e.g., glitter, shredded paper, talcum or other powders), tactile deterrents (e.g., irritant powder or liquids), or combinations thereof. The distraction elements are automatically discharged or thrown from the pouch 400 as the restraint system 200 is deployed (as described below) and the sling element 206 pulled taut. These distraction elements 418 temporarily distract or disorient a target individual, giving the user or users a greater immediate advantage in subduing the target with the restraint system 200.

Additionally, other components may be automatically deployed to the advantage of the users. For example, deployment of the restraint system 200 may activate high-intensity constant or strobe lights, cameras, high-pitch audible alarms, mace or chemical irritants, liquids or foams (to cause the target to lose traction), or other components. In these examples, the system 200 is in a rolled or stored configuration, elements requiring battery power (lights, alarms) may be disengaged by a contact breaking the power circuit. Opening of the restraint system 200 may break or remove this contact, thus providing power to the distraction element. Discharged chemicals, liquids, or foams may be stored in pressurized canisters on the inner face of the sling element. Deployment of the system 200 may activate a discharge mechanism (e.g., pull a pin on a pressurized canister) to discharge the contents of the canister. In general, distraction elements should be located on the inner face of the restraint system 200 so as to be directed at the target during system 200 deployment.

FIG. 5 depicts an enlarged view of the pouch 400 attached to the pouch securement areas 302, 304 of the restraint system 200, during discharge of the distraction element 418. As can be seen, the pouch 400 is located towards a top of the restraint system 200, thus allowing an optional second distraction element to be utilized below. For example, pouches located at the upper portion of the restraint system 200 may discharge distraction elements (e.g., irritant powders) to temporarily incapacitate or blind a target, while pouches located at the lower portion of the restraint system 200 may discharge distraction elements (e.g., lubricants) to cause the target to fall. A pouch 400 may be reusable or single-use. Reusable pouches 400 may be refilled as needed with distraction elements 418, e.g., by filling the pouch 400 from an upper portion thereof, via a storage bag or vessel 420.

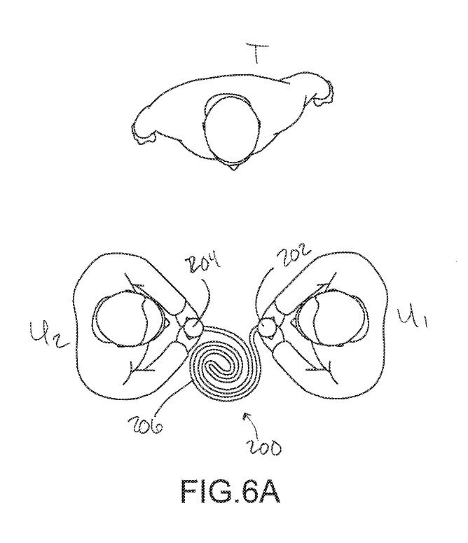

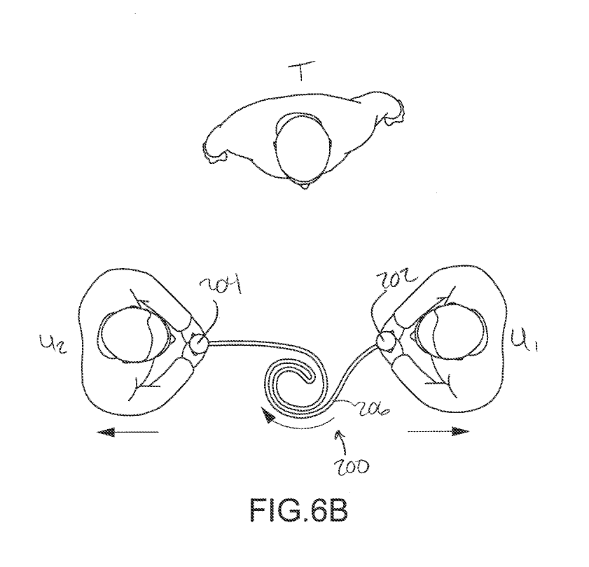

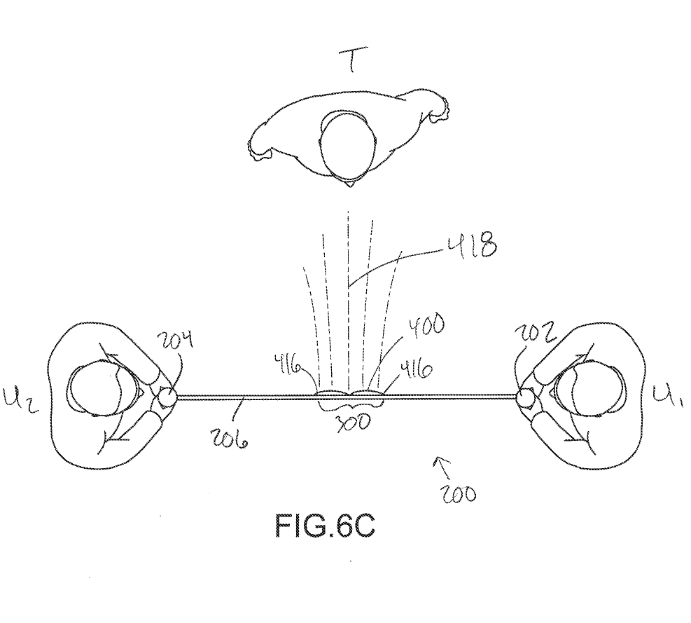

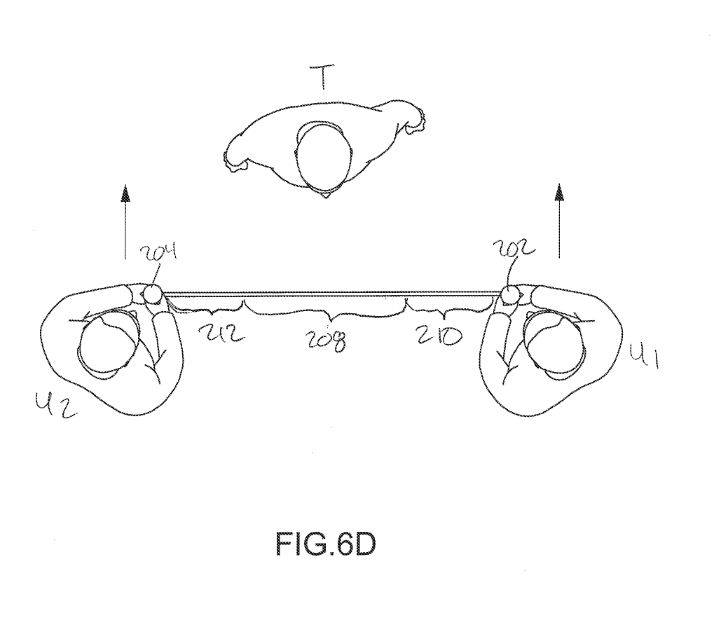

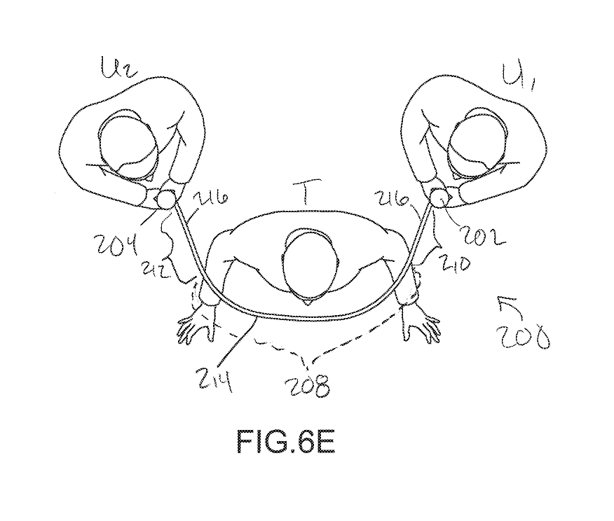

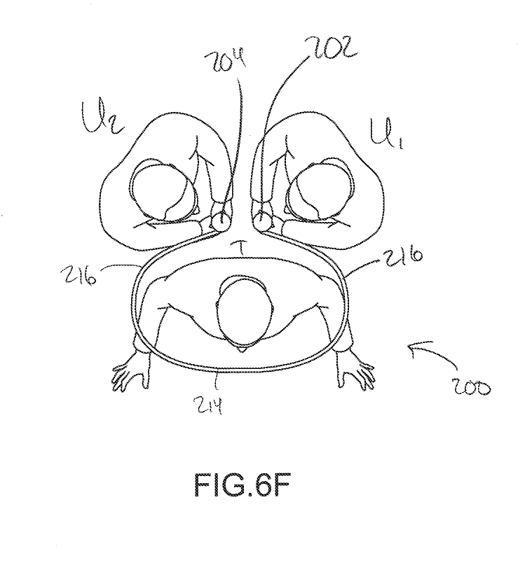

A sequence for using a restraint system 200 against a target T is depicted in FIGS. 6A-6F. In FIG. 6A, the system 200 is in a rolled closed position. To be properly deployed, the restraint system 200 must first be stored in the rolled closed position. To configure the system 200 into the rolled closed position, the restraint system 200 is first folded, such that the handles 202, 204 are proximate each other and pouch (not depicted) is located proximate a fold of the sling element 206. The sling element 206 is then rolled from the fold towards the handles 202, 204, such that both handles 202, 204 are exposed and accessible prior to deployment. In this rolled or stored position, the system 200 may be secured with one or more ties or closure elements (not shown). Additionally or alternatively, one or more mechanical devices or clips may be wrapped around the system 200 to maintain the rolled position. To deploy the restraint system 200, a first user U₁ grasps a first handle 202 and a second user U₂ grasps a second handle 204. Any ties, closures, or other mechanical elements holding the rolled system 200 are removed or disengaged. The handles 202, 204 are then forcibly pulled away from each other, as depicted in FIG. 6B. This may be accomplished by one user pulling rapidly away from the other, immobile user, or by both users pulling their respective handle 202, 204 away from the other. As the handles 202, 204 move away from each other, the sling element 206 unrolls therebetween. Just prior to the sling element 206 being pulled taut, the edges 416 of the pouch 400 begin to separate from each other, since their outer faces are secured to the pouch securement area 300.

As the sling element 206 is pulled taut, the pouch 400 opens completely, and the contents (the distraction element 418) inside are forcibly expelled therefrom toward the target T, as depicted in FIG. 6C. In the depicted embodiment, the distraction element 418 is an irritant powder). As the distraction element 418 is directed at the target T, the target T becomes disoriented. Of course, incorporation of the pouch 400 and distraction element 418 is entirely optional. The users U₁, U₂ may then advance on the target T, as depicted in FIG. 6D. Any responsive behavior of the target T is not critical to the use of the system 200. If the target T is able, the target T may advance on the restraint device 200 (as described above, believing the central webbing portion to be compromised or weak, for example). Alternatively, the target T may remain stationary due to the effects of the distraction element, stress, indecision, or for other reasons. Regardless, as the users U₁, U₂ approach the target T, the central portion 208 of the restraint system 200 first contacts the target T. At this position, hands and/or limbs may pass through the webbing 214 if the target T has extended his arms to protect himself, as in FIG. 6E. The users U₁, U₂ then close around the rear of the target T, coming together as in FIG. 6F. At this position, the handles 202, 204 may be secured to each other with straps or other elements or simply held by the users U₁, U₂ to completely contain the target T. From this position, further restraints or shackles may be applied to the target T, or medicines or sedatives administered.

The restraint systems described herein may be used for other purposes in addition to restraining one or more individuals. As described above, the systems may be used as a portable fence or screen. Single or multiple restraint systems may be used to push or corral one or more individuals for crowd control or other purposes. The restraint systems may also be used as a hammock, field litter, or other implement for supporting a reclining person. Different types of materials may be used, depending on application and expected use of the system. For example, nylon webbing having a width of 1 or 2 inches may be utilized. In another embodiment, the webbing located at the central portion may be clear or translucent vinyl, which may help convince a target that the central portion is weak, compromised, or even non-existent. In that regard, clear or translucent vinyl or other plastic may be used for the entire system, both the central portion and outer portions. Other materials may include polyethylene or other robust plastics. The handles may be made of plastic or metal, or a combination thereof (i.e., a plastic overmolded onto a metal core). Hollow or solid materials may be utilized. Acceptable plastics may include PVC, ABS, or fiber reinforced plastics. Acceptable metals may include steel, aluminum, or other materials. These are merely exemplary and may be modified as required or desired for a particular application. A particular commercial embodiment of the system depicted herein has a length L of about six feet and a height of about two feet. Systems having other dimensions are contemplated. For example, other embodiments having a length greater than six feet may be particularly useful for crowd control/portable barrier applications. Having additional handles (such as the third handle 124 in FIG. 1) located at intervals along the length of the sling material may be desirable for longer systems. Additionally, embodiments having heights near that of an average person may be more advantageous for barrier applications.

While there have been described herein what are to be considered exemplary and preferred embodiments of the present technology, other modifications of the technology will become apparent to those skilled in the art from the teachings herein. The particular methods of manufacture and geometries disclosed herein are exemplary in nature and are not to be considered limiting. It is therefore desired to be secured in the appended claims all such modifications as fall within the spirit and scope of the technology. Accordingly, what is desired to be secured by Letters Patent is the technology as defined and differentiated in the following claims, and all equivalents.

What is claimed is:

1. A restraint system comprising:
a first handle and a second handle; and
a sling element connected at a first edge to the first handle and at a second edge to the second handle, the sling element comprising a webbed material.

2. The restraint system of claim 1, wherein the sling element further comprises a first non-webbed section connected to the first handle and the webbed material.

3. The restraint system of claim 2, wherein the sling element further comprises a second non-webbed section connected to the second handle and the webbed material.

4. The restraint system of claim 1, wherein the sling element comprises an outer face and an inner face.

5. The restraint system of claim 4, wherein the sling element inner face comprises a pouch securement area comprising a first securement area and a second securement area.

6. The restraint system of claim 5, further comprising a pouch fixed to the pouch securement area.

7. The restraint system of claim 6, wherein the pouch comprises:
a first securement portion;
a second securement portion, wherein each of the first securement portion and the second securement portion comprise an outer face and an inner face; and
a fold located between the first securement portion and the second securement portion.

8. The restraint system of claim 7, wherein the outer face of the first securement portion is connected to the first securement area and wherein the outer face of the second securement portion is connected to the second securement area.

9. The restraint system of claim 8, wherein the inner face of the first securement portion and the inner face of the second securement portion are adapted to be connected to each other when the restraint system is in a stored position and disconnected from each other when the restraint system is in a deployed position.

10. The restraint system of claim 9, wherein the pouch is adapted to receive a distraction element, wherein the distraction element is adapted to be discharged from the pouch when the restraint system is in the deployed position.

11. The restraint system of claim 10, wherein the distraction element comprises at least one of glitter, frag, and powder.

12. The restraint system of claim 9, wherein when in the deployed position, the sling element is taut between the first handle and the second handle.

13. The restraint system of claim 4, further comprising a third handle connected to the sling element outer face.

14. The restraint system of claim 4, wherein the sling element outer face comprises at least one accessory fixture.

15. The restraint system of claim 14, wherein the accessory fixture comprises at least one of a snap connection, a pocket, an elastic element, and a loop.

16. The restraint system of claim 1, further comprising a connecting element proximate the first handle, wherein the connecting element is adapted to connect the restraint system to a second restraint system.

17. A method of deploying a restraint system comprising a first handle, a second handle, and a sling element strung between the first handle and the second handle, the method comprising:
pulling the first handle in a direction substantially away from the second handle, such that the sling element is made taut between the first handle and the second handle.

18. The method of claim 17, wherein the first handle is grasped by a first user and the second handle is grasped by a second user.

19. The method of claim 17, wherein the restraint system automatically discharges a distraction element as the sling element is made taut.

20. The method of claim 17, further comprising wrapping the restraint system around at least one target.
